(19) United States
(12) Patent Application Publication
(10) Pub. No.: US 2021/0169340 A1
(43) Pub. Date: Jun. 10, 2021

(54) Method for Realizing Arbitrary Ultrasonic Field

(71) Applicant: SOUTH CHINA UNIVERSITY OF TECHNOLOGY, Guangzhou, Guangdong (CN)

(72) Inventors: Honglian Guo, Guangzhou, Guangdong (CN); Hongyan Yuan, Guangzhou, Guangdong (CN); Feng Li, Guangzhou, Guangdong (CN); Baoqin Chen, Guangzhou, Guangdong (CN); Tian Wei, Guangzhou, Guangdong (CN)

(21) Appl. No.: 16/762,259

(22) PCT Filed: Oct. 26, 2018
(86) PCT No.: PCT/CN2018/112113

§ 371 Date: May 7, 2020

(30) Foreign Application Priority Data
Nov. 7, 2017 (CN) 201711082071.0

(51) Int. Cl. A61B 5/00

(52) U.S. Cl. CPC A61B 5/0095

(57) ABSTRACT

The invention discloses a method for realizing an arbitrary ultrasonic field. The method uses a pulsed laser generated by a pulsed laser machine, modulated into a specific two-dimensional or three-dimensional light field distribution by a phase-type spatial light modulator, and the specific distribution of light acts on photoacoustic media. Due to the photoacoustic effect, a sound field distribution corresponding to the light field will be generated in the photoacoustic medium. The sound field constitutes a two-dimensional or three-dimensional spatial sound source. The sound waves emitted by the sound source are transmitted through a certain distance. The expected ultrasonic field distribution can be formed on a surface or inside of samples. The invention can transform the coherent ultrasonic field distribution formed on the surface or inside of the target object, simply by adjusting the phase distribution diagram input to the spatial light modulator, which has great flexibility.

### RELATED APPLICATIONS

This application is a U.S. national phase application, claiming priority under 35 U.S.C. § 371 to PCT application PCT/CN2018/112113, filed Oct. 26, 2018, claiming priority to Chinese Patent Application No. 201711082071.0, filed on Nov. 7, 2017. The contents of these applications are incorporated herein by reference in their entireties.

### FIELD OF THE INVENTION

The invention relates to the field of ultrasound field, and in particular to a method for realizing arbitrary ultrasound field.

### BACKGROUND OF THE INVENTION

Not only is ultrasound imaging an important means of clinical medical imaging testing, it is also an important method of industrial non-destructive testing. Scanning acoustic holography introduces the interference principle of optical holography into the field of acoustics, and uses array transducers or array detectors to directly measure the amplitude and phase of an object wave diffraction field, thereby reconstructing a visible image of the object. Not only can ultrasound be used as an image information acquisition tool, it takes advantages of strong ultrasonic penetration and good directional performance, and can also be a tool for energy transmission. It can be used for cleaning, crushing stones, acoustic tweezers, etc., and has applications in medicine, military and industry. Ultrasound imaging usually uses piezoelectric transducer arrays to excite and detect ultrasonic waves. The number of array units directly determines the imaging quality: the more the number of array units, the larger the spatial bandwidth product and the clearer the imaging. However, increase in the number of array units also increases size and complexity of the system hardware.

In the prior art, some researchers used a phase array (12×12) composed of two-dimensional plastic materials to demonstrate the generation of arbitrary ultrasound fields by air acoustic holography. Although this method demonstrates the concept of acoustic holography well, due to its large unit size and that it cannot actively modulate the phase, there is still a long way to go from application. Some researchers used 3D printing to generate an acoustic holographic plate, and then used the sound waves generated by unit plane acoustic wave generator to pass through the holographic plate, thus achieving a pre-set sound field graphics. There are also researchers who further used 3D printing technology to process photoacoustic materials with special surface topography, and used pulsed laser to excite the photoacoustic materials to achieve the distribution of arbitrary ultrasonic field. The advantages of these two methods of realizing sound field control based on 3D printing technology compared with the previous realization of arbitrary ultrasonic field are: the use of 3D printed media with special surface topography replaces the previous complex array transducer, and the sound field quality is not affected by the limit of the number of units of the transducer. However, the disadvantage of this method is that the material of the acoustic holographic plate corresponds to a certain pattern of ultrasonic field. If the ultrasonic field needs to be changed, the holographic plate must be newly manufactured, which greatly limits its application.

Photoacoustic imaging is a non-destructive biophotonic imaging method based on the difference in optical absorption within biological tissues and using ultrasound as a medium. It combines the advantages of pure optical imaging with high contrast characteristics and pure ultrasound imaging with high penetration depth characteristics, which can provide high contrast and high-resolution tissue images that have wide application prospects in the field of biomedical clinical diagnosis and imaging of body tissue structure and function. Yao et al. used galvanometers to achieve fast photoacoustic scanning microscopic imaging. Galvanometers and other movable parts not only increase the complexity of the system but also reduce the photoacoustic coupling efficiency. Based on this, we propose a spatial light modulator to modulate the pulsed laser, then to act on the photoacoustic medium to achieve a dynamic construction method of arbitrary ultrasonic field.

### SUMMARY OF INVENTION

The purpose of the present invention is to provide a method for realizing an arbitrary ultrasonic field in response to the above-mentioned deficiencies of the prior art. The method uses a spatial light modulator to phase modulate a pulsed laser to generate a required spatial light field distribution. The spatially modulated pulsed laser further acts on a medium. Due to the photoacoustic effect, the pulsed laser will produce a specific pressure distribution, thereby achieving arbitrary ultrasonic field distribution in the medium.

The object of the present invention can be achieved by the following technical solutions:

A method for realizing an arbitrary ultrasonic field, the method comprising the following steps:

S1. irradiates on a spatial light modulator a pulsed laser emitted by a pulsed laser machine expanded by an optical beam expander;

S2. a computer calculates and generates a corresponding phase distribution diagram according to a design goal and inputs it to the spatial light modulator; after a phase modulation of the pulsed laser irradiated on the spatial light modulator, forms a light field with a target phase distribution pattern at a focal plane through an optical lens;

S3. the light field with the target phase distribution pattern generated in step S2 acts on a photoacoustic medium; due to a photoacoustic effect, a sound field distribution corresponding to the light field is generated inside the photoacoustic medium, and the sound field constitutes a two-dimensional or three-dimensional spatial sound source; sound waves emitted by the spatial sound source are transmitted through a certain distance to form an expected coherent ultrasound field distribution on a surface or inside of a target object.

Further, the spatial light modulator is a phase-type spatial light modulator.

Further, the sound field in the photoacoustic medium acting on the target object can be used for a selective high-throughput imaging of biological tissues; and can also be used for capturing and directional transportation of granular objects.

Further, by adjusting the phase distribution diagram input to the spatial light modulator, a coherent ultrasonic field distribution formed on the surface or inside of the target object is changeable at any time.

Compared with the prior art, the present invention has the following advantages and beneficial effects:

The method of the present invention does not realize the distribution of arbitrary ultrasonic field through a traditional ultrasonic transducer or a complex ultrasonic transducer, but controls the sound field distribution through a light field, and the light field distribution is realized by a spatial light modulator. Not only does it ensure the spatial arbitrary adjustment of the sound field, it also realizes the dynamic change of the sound field. Compared with traditional ultrasonic transducers or complex ultrasonic transducers, it has great flexibility and convenience, and is expected to have direct application in ultrasound imaging, sonic control and other fields.

### BRIEF DESCRIPTION OF THE FIGURES

FIG. 1 is an optical path diagram of an arbitrary ultrasonic field method according to an embodiment of the present invention.

### DETAILED DESCRIPTION OF THE INVENTION

The present invention will be described in further detail below with reference to examples and drawings, but the embodiments of the present invention are not limited thereto.

### Example

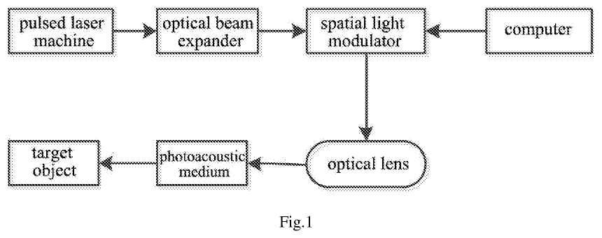

This embodiment provides a method for realizing an arbitrary ultrasonic field. The optical path diagram for implementing the method is shown in FIG. 1 and includes the following steps:

S1. irradiates on a phase-type spatial light modulator a pulsed laser emitted by a pulsed laser machine expanded by an optical beam expander;

S2. a computer calculates and generates a corresponding phase distribution diagram according to a design goal and inputs it to the phase-type spatial light modulator; controls the pulsed laser irradiated on the spatial light modulator after a phase modulation, forms a light field with a target phase distribution pattern at a focal plane through an optical lens;

S3. the light field with the target phase distribution pattern generated in step S2 acts on a photoacoustic medium; due to a photoacoustic effect, a sound field distribution corresponding to the light field is generated inside the photoacoustic medium, and the sound field constitutes a two-dimensional or three-dimensional spatial sound source; sound waves emitted by the spatial sound source are transmitted through a certain distance to form an expected ultrasound field distribution on a surface or inside of a target object.

In particular, the sound field in the photoacoustic medium acting on the target object can be used for a selective high-throughput imaging of biological tissues; and can also be used for capturing and directional transportation of granular objects.

Further, by adjusting the phase distribution diagram input to the spatial light modulator, an ultrasonic field distribution formed on the surface or inside of the target object is changeable.

The above is only the preferred embodiment of the invention patent, and the protection scope of the invention patent is not limited to this. Equivalent replacements and changes according to the technical solution of the invention patent and the concept of the invention patent etc., within the scope disclosed by the invention patent, by any person skilled in the art in the technical field, belong to the protection scope of the invention patent.

1. A method for realizing an arbitrary ultrasonic field, characterized in that, the method comprising the following steps:
S1. irradiates on a spatial light modulator a pulsed laser emitted by a pulsed laser machine expanded by an optical beam expander;
S2. a computer calculates and generates a corresponding phase distribution diagram according to a design goal and inputs it to the spatial light modulator; after a phase modulation of the pulsed laser irradiated on the spatial light modulator, forms a light field with a target phase distribution pattern at a focal plane through an optical lens;
S3. the light field with the target phase distribution pattern generated in step S2 acts on a photoacoustic medium; due to a photoacoustic effect, a sound field distribution corresponding to the light field is generated inside the photoacoustic medium, and the sound field constitutes a two-dimensional or three-dimensional spatial sound source; sound waves emitted by the spatial sound source are transmitted through a certain distance to form an expected coherent ultrasound field distribution on a surface or inside of a target object.

2. The method for realizing an arbitrary ultrasonic field according to claim 1, characterized in that, the spatial light modulator is a phase-type spatial light modulator.

3. The method for realizing an arbitrary ultrasonic field according to claim 1, characterized in that, the sound field in the photoacoustic medium acting on the target object can be used for a selective high-throughput imaging of biological tissues; and can also be used for capturing and directional transportation of granular objects.

4. The method for realizing an arbitrary ultrasonic field according to claim 1, characterized in that, by adjusting the phase distribution diagram input to the spatial light modulator, a coherent ultrasonic field distribution formed on the surface or inside of the target object is changeable at any time.
